(19) United States
(12) Patent Application Publication
(10) Pub. No.: US 2019/0083285 A1
(43) Pub. Date: Mar. 21, 2019

(54) FLEXIBLE PROSTHETIC LINER AND PROSTHETIC SHAFT SYSTEM

(71) Applicant: OTTOBOCK SE & CO. KGAA, Duderstadt (DE)

(72) Inventors: Martin HILLMANN, Duderstadt (DE); Michael RAUSCH, Brehme (DE); Roger ROST, Gottingen (DE); Luder MOSLER, Duderstadt (DE)

(73) Assignee: OTTOBOCK SE & CO. KGAA, Duderstadt (DE)

(21) Appl. No.: 16/085,369

(22) PCT Filed: Mar. 24, 2017
(86) PCT No.: PCT/EP2017/057067

§ 371 Date: Sep. 14, 2018

(30) Foreign Application Priority Data
Mar. 24, 2016 (DE) 10 2016 105 615.4

(51) Int. Cl. A61F 2/78; A61F 2/80

(52) U.S. Cl. CPC A61F 2/7812; A61F 2002/5083; A61F 2/80

(57) ABSTRACT

A flexible prosthetic liner having a proximal access opening for a stump of a limb and a distal end region as well as a flexible tension member which is arranged in the distal 5 end region on the prosthetic liner. The tension member is detachably fastened to the prosthetic liner by means of a closure system that can be actuated without a tool. A prosthetic shaft system may be formed by such a prosthetic liner and a prosthetic shaft.

The invention relates to a flexible prosthetic liner with a proximal access opening for a stump of a limb, and a distal end and also a flexible tensioning means which is arranged on the prosthetic liner in a distal end region. The invention likewise relates to a prosthesis socket system with such a prosthetic liner, and a prosthesis socket which has a proximal access opening, a side wall at least partially circumferentially surrounding the prosthetic liner, and a distal closing region.

U.S. Pat. No. 6,793,682 B2 discloses a locking system with a flexible liner which surrounds a limb stump. An outlet at the lower distal end is arranged to the liner. A first strap is arranged at a proximal, i.e. upper, end of the prosthetic liner, and a loop is formed on said first strap. A second strap is screwed on the liner centrally at the distal end thereof and is brought out of a side wall of a prosthesis socket. A hook and loop fastener is arranged on the second strap, and therefore, after the free end of the second strap is passed through the loop and turned back over on itself, the second strap can be fixed. For removing the stump from the prosthesis socket or for taking off the prosthesis socket, the second strap is released, pulled out of the loop on the first strap and brought out of the prosthesis socket together with the first strap. The free strap end is pulled here out of the pass-through opening in the distal socket region.

In particular for geriatric patients, the inserting of the free strap end through the prosthesis socket and the passage opening in the distal prosthesis socket region is cumbersome.

It is an object of the present invention to provide an improved prosthetic liner and an improved prosthesis socket system consisting of prosthetic liner and prosthesis socket.

This object is achieved according to the invention by a flexible prosthetic liner with the features of the main claim and a prosthesis socket system with the features of the further independent claim. Advantageous refinements and developments of the invention are explained in more detail in the dependent claims, the description and in the figures.

The flexible prosthetic liner according to the invention with a proximal access opening for a stump of a limb, with a distal end region and also a flexible tensioning means that is arranged on the prosthetic liner in the distal end region makes provision for the tensioning means to be fastened releasably to the prosthetic liner via a closure system which is actuable without a tool. By means of the fastening of the tensioning means to, and separating of same from, the distal end of the prosthetic liner without a tool, it is possible for the tensioning means, which is preferably in the form of an inelastic strap, to remain on a prosthesis socket when the prosthesis socket is to be removed and a patient removes his stump from the prosthesis socket. The cumbersome insertion of the free end of the tensioning means is thus superfluous, and, for fitting the prosthesis socket, a patient merely has to connect the prosthetic liner to the tensioning means via the closure system and actuate the tensioning means, which continues to be located within the prosthesis socket, in order firstly to facilitate the insertion into the prosthesis socket and secondly to ensure locking against an axial pulling-out movement counter to the access direction. Furthermore, by means of the separation of liner and tensioning means, one of the two components may be exchanged if required without the component which is still usable having to be changed.

The tensioning means is in particular fastened to the prosthetic liner in a form-fitting manner; additionally or alternatively, a force-fitting, in particular magnetic locking or at least securing of the form-fitting locking by force-fitting, in particular magnetic locking can also be achieved. Magnetic forces can additionally serve to make insertion, approach and/or orientation of the connecting components easier.

The tensioning means can have a first form-fitting element which enter into engagement with a second form-fitting element, which is arranged permanently on the prosthetic liner, in order thus to produce a secure connection, which can be produced in a simple manner, between the tensioning means and the prosthetic liner. The form-fitting elements have at least one undercut and interact in such a manner that the closure system is in the form of a snap-action connection, a hook and loop fastener, a press stud, a sliding insert, a clip connection, a loop and/or a quarter-turn fastener. The form-fitting connection between the two form-fitting elements can optionally be secured by additional securing, for example rotational securing on the basis of magnetic locking elements, a rotational and sliding movement, a reduction in length or reduction in circumference or a combination thereof such that inadvertent unlocking is impossible. Securing against unlocking can also be arranged or formed within the prosthesis socket, for example by fitting the closure system into a corresponding recess such that a relative movement between the form-fitting elements, which are in engagement, after the insertion into a prosthesis socket is no longer possible.

In addition to the form-fitting elements, the closure system can have magnetic securing in order to apply a further holding force. With a magnetic element, orientation of the closure elements with respect to one another can be ensured; it is also possible by means of a magnetic component to bring about a defined relative movement between the two form-fitting elements such that locking also takes place counter to the original insertion direction. Said locking can be released by an actuating element or by overcoming the magnetic securing on the basis of a rotational movement, sliding movement, pressing movement or the like.

The closure system and in particular the form-fitting elements used therein preferably block a movement between the tensioning means and the prosthetic liner counter to the insertion direction, and therefore it is ensured that the prosthetic liner is securely held and fixed in the distal region on a prosthesis socket when the tensioning means is secured to the prosthesis socket.

The form-fitting element can also block shifting of the form-fitting elements with respect to one another, in at least one direction perpendicular to the pulling-out direction, for example by means of a sliding device with an end stop, a dovetail guide, a snap-action connection or by other suitable undercuts. The spatial orientation of the tensioning means on the prosthetic liner is thereby ensured, in particular when all of the translatory degrees of freedom are blocked by the closure system, which is basically provided, for example, in the case of spherical snap-action connections or press stud closures.

A form-fitting and/or force-fitting locking device for locking the prosthetic liner to a prosthesis socket can be arranged or formed at the distal end of the prosthetic liner, said locking device acting additionally to the tensioning means in order to bring about further securing of the liner on a prosthesis socket.

In order to prevent the free end of the tensioning means from unintentionally sliding out of the prosthesis socket, a preferably removable retaining device can be arranged at the end lying opposite the closure system, said retaining device having such dimensions that the retaining device and the tensioning means cannot pass through a passage opening in the prosthesis socket. After the tensioning means is passed for the first time through the passage opening, the retaining element is fastened to the tensioning means, for example in the form of a blocking element which can be clipped on or is fastenable via a hook and loop fastener. The blocking element prevents the tensioning means from being inadvertently separated from the prosthesis socket. At the other end, that part of the closure system which is fastened to the tensioning means prevents slipping out of the prosthesis socket.

The prosthesis socket system according to the invention with a prosthetic liner, as has been described above, and with a prosthesis socket with a proximal access opening, a side wall at least partially circumferentially surrounding a stump, and with a distal closing region, provides a passage opening in the distal closing region for the tensioning means arranged on the distal end region of the prosthetic liner, and therefore both securing against pulling out of the prosthetic liner and therefore of the stump from the prosthesis socket and defining of the rotational orientation of the prosthesis socket on the prosthetic liner and therefore on the prosthetic stump can be ensured.

The closing region of the prosthesis socket can have a supporting device with a recess for the closure system, and therefore the closure system which is located in the distal end region of the prosthetic liner does not lead to undesirable increases in pressure on the stump. The supporting device is preferably dimensionally stable and can be formed in the manner of a bowl. The recess can serve at the same time as securing for securing the tensioning means on the prosthetic liner, for example by sliding out of a form-fitting element being prevented by an appropriately positioned side wall.

A fixing device for the tensioning means arranged in the distal end region of the prosthetic liner, for example a buckle, a hook and loop fastener or another form-fitting means for fixing the tensioning means to the prosthesis socket, can be provided on the outer side of the side wall of the prosthesis socket. Alternatively, a deflecting element can be fixed to the side wall such that, by inserting the tensioning element into the deflecting element and deflecting and fixing same on itself, for example by means of a hook and loop fastener, a buckle or a hook, the tensioning means is fixed to the prosthesis socket.

In the prosthesis socket, a window can be provided in the proximal region of the side wall as a second pass-through opening for a second strap or for a second tensioning means which is fastened to the proximal end region of the flexible prosthetic liner, and therefore the two tensioning means are also fastenable to each other, for example via a hook and loop fastener, press studs, a buckle or the like.

The pass-through opening for the passage of the first tensioning means, which is secured in the distal end region of the prosthetic liner on the latter via the closure system, is preferably oriented perpendicular to the insertion direction of the stump and prosthetic liner in the prosthesis socket. As a result, after the tensioning means is fitted and secured, tensile forces are produced which act in a horizontal plane. The tensile forces load the closure system with forces within the horizontal plane, and therefore, for example by configuration of the closure system as a form-fitting device with a sliding component, the forces in the horizontal plane can easily be absorbed by means of a stop. At the same time, it can be prevented by the stop that the closure system is inadvertently opened. With the closure system being simultaneously configured with a form-fitting component which blocks a movement in the pulling-out direction, the prosthetic liner is secured in the prosthesis socket.

The pass-through opening is preferably arranged laterally such that the tensioning means can easily be led out on the outer side, actuated and secured thereto.

Exemplary embodiments of the invention will be explained in more detail below with reference to the attached figures. The same reference signs denote identical components. In the figures:

FIG. 1 shows the components of a prosthesis socket system in a partially mounted state;

FIG. 2 shows a detailed view of a closure system;

FIG. 2a shows a partial view of a form-fitting element;

FIG. 3 shows a variant of the closure system;

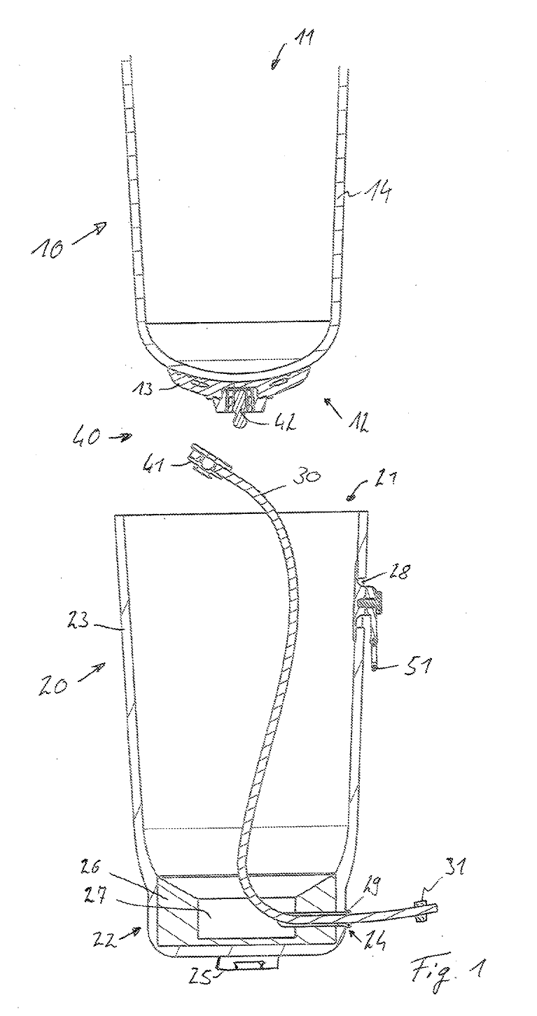

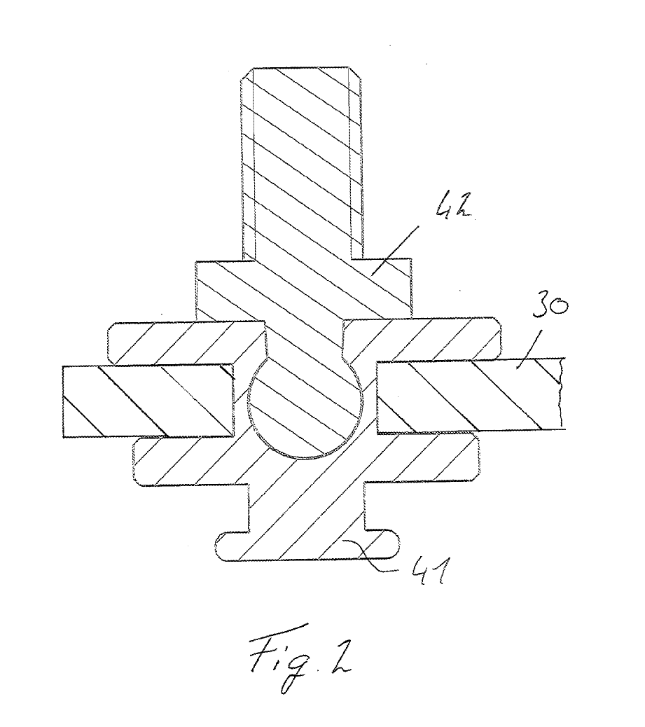

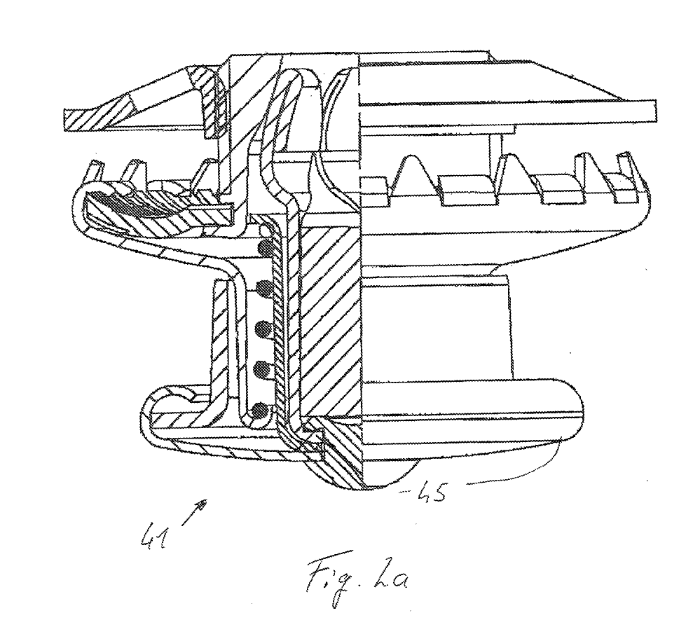

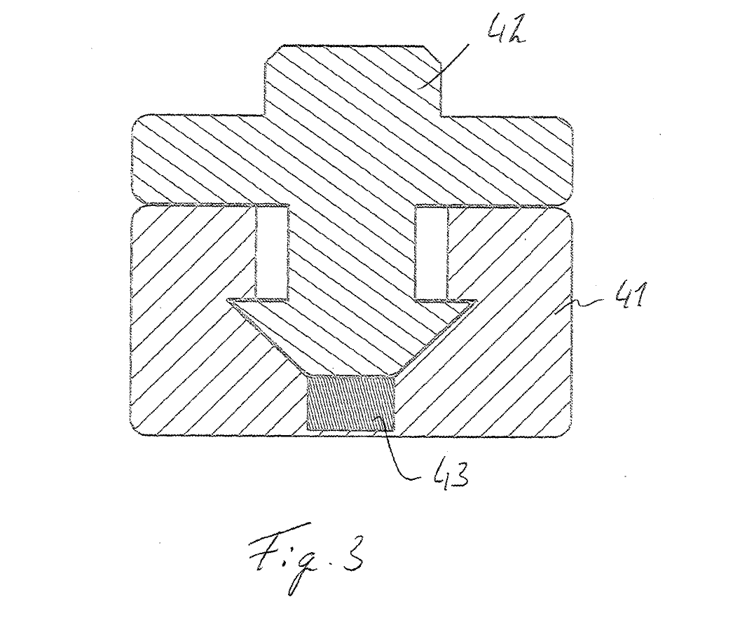

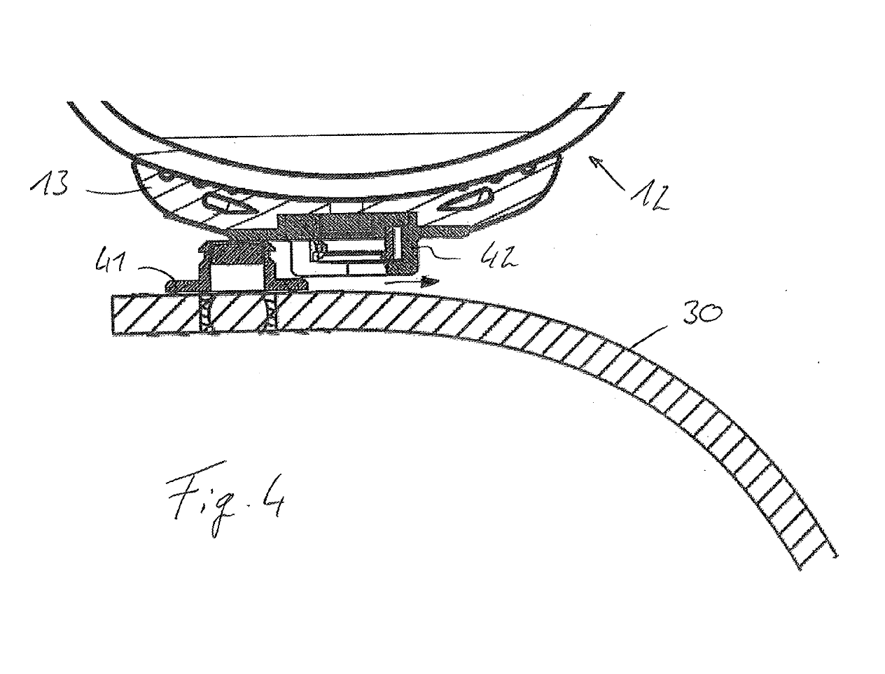

FIG. 4 shows a detailed illustration of a prosthetic liner with tensioning means before assembly;

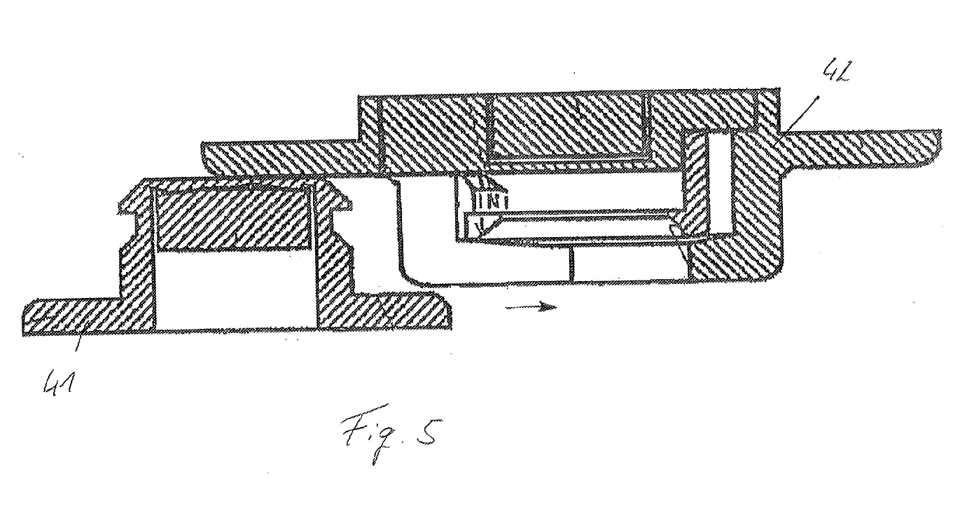

FIG. 5 shows a detailed view of FIG. 4;

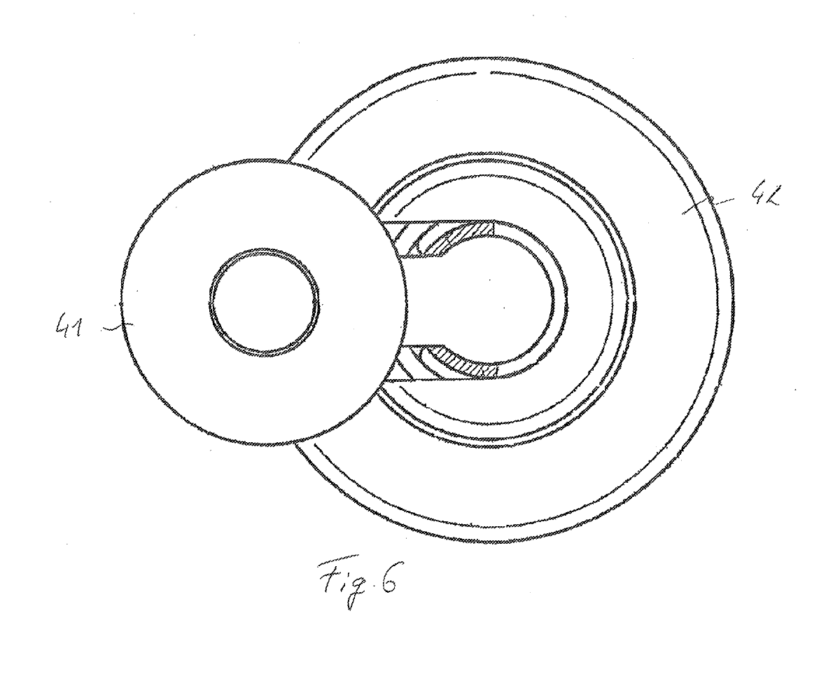

FIG. 6 shows a top view of a closure system according to FIG. 5;

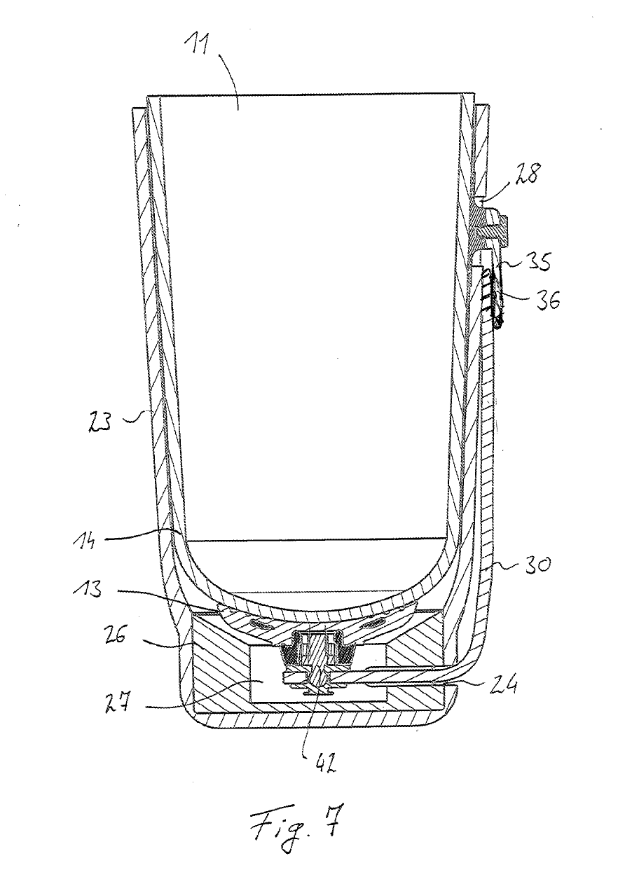

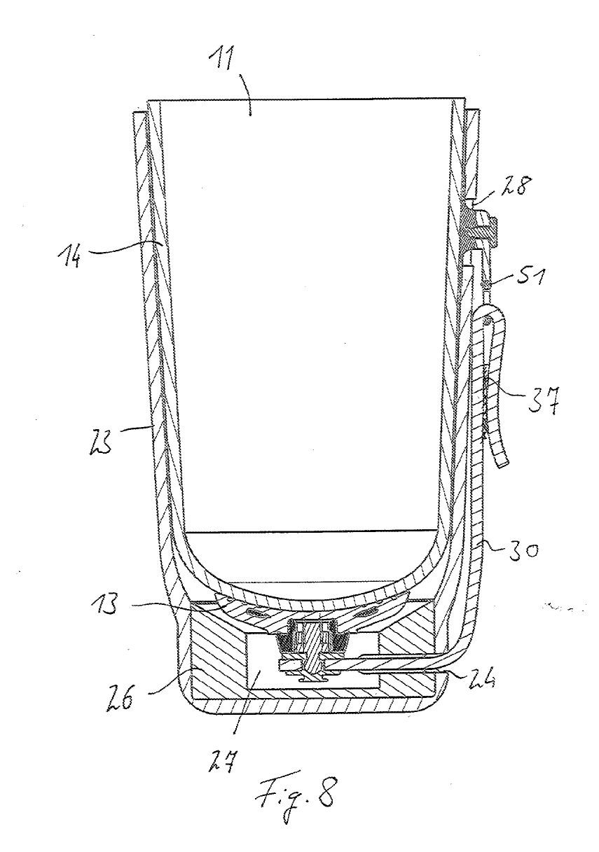

FIGS. 7 and 8 show variants of the fitted prosthesis socket system in a sectional view;

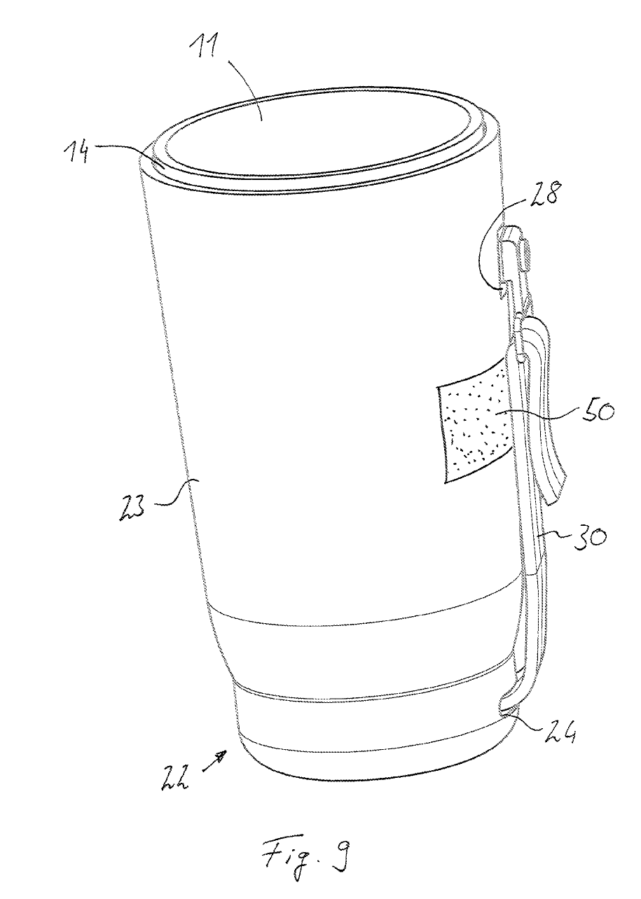

FIG. 9 shows a perspective illustration of a fitted prosthesis socket system; and

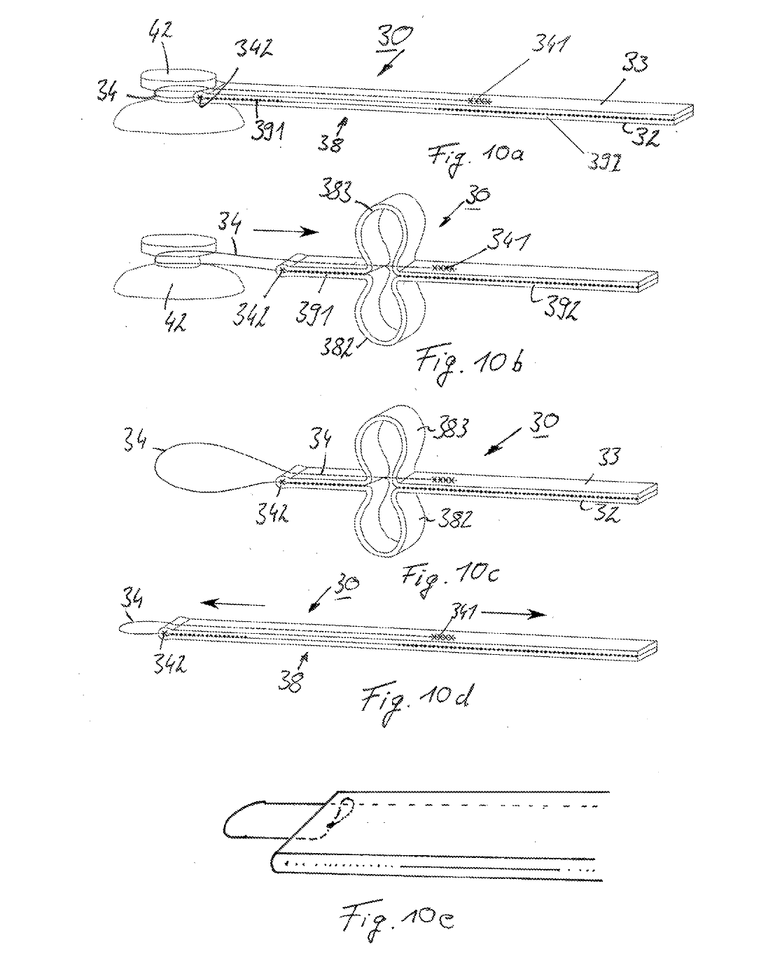

FIGS. 10a to 10e show individual illustrations of a tensioning means.

FIG. 1 shows a prosthesis socket system with a flexible prosthetic liner 10 which has a proximal access opening 11 for a stump of a limb, and also a distal end region 12. The prosthetic liner 10 is preferably formed in a closed manner in the distal end region 12, and also the side wall 14, which reaches from the distal end region 12 as far as the access opening 11, can be formed with a closed wall, and therefore the prosthetic liner 10 can be placed around the entire circumference of the stump (not illustrated) of a limb. The prosthetic liner 10 is preferably composed of a silicone or another flexible or optionally elastic polymer in the region of the side wall. In principle, it is also possible for a different material than the basic material to be applied or incorporated on or in the inner side or on or in the outer side of the prosthetic liner 10; a textile coating can also be arranged on one of the surfaces of the prosthetic liner 10.

A dimensionally stable closing cap 13 which is flexible to a limited extent and forms the distal closure of the prosthetic liner 10 is arranged at the distal end region 12, which is closed in the exemplary embodiment, of the prosthetic liner 10. The closing cap 13 serves for stabilizing the distal end region 12 and for forming a resting surface for the end of the stump. A second form-fitting element 42 is arranged, screwed in the exemplary embodiment shown, within the distal closing cap 13 and can be brought into engagement with a first form-fitting element 41, which is fastened to a flexible tensioning means 30, which is preferably rigid under tension, in the form of a strap or a cable.

The first form-fitting element 41 is secured to the tensioning means 30 in a form-fitting manner, but other types of fastening are also possible. The first form-fitting element 41 together with the second form-fitting element 42 forms a closure system 40, wherein the two form-fitting elements 41, 42 are separable from each other and securable to each other without a tool, and therefore the tensioning means 30 can be rapidly fastened to the prosthetic liner 10 or separated therefrom.

The tensioning means 30 is inserted in a prosthesis socket 20. The prosthesis socket 20 has a proximal access opening 21, a distal closing region 22 and an at least partially encircling side wall 23 via which a receiving space for the prosthetic liner 10 together with the stump is formed. The prosthesis socket 20 can be manufactured individually for the user, for example by taking impressions of the stump and adjusting a single-part prosthesis socket which is closed in the circumferential direction and follows the contour of the stump. The side wall 23 here can have a closed cross section, at least over most of the longitudinal extent or height of the prosthesis socket. Windows 28 can also be formed in the prosthesis socket 20 or the side wall 23. It is likewise possible for the prosthesis socket 20 to be composed of a plurality of segments which are integrally formed on or fastened to the distal closing region 22 and, fitted state, form a dimensionally stable prosthesis socket 20. The prosthesis socket 20 serves for receiving the prosthetic liner 10 with the stump and the arrangement and securing of further prosthetic components, for example prosthetic knee joints, prosthetic feet or prosthetic hands.

The prosthesis socket 20 in the example illustrated has a dimensionally stable supporting device 26 in the distal end region 22, said supporting device being arranged within the prosthesis socket 20. In principle, it is also possible to arrange the dimensionally stable supporting device 26 on the outer side. The supporting device 26 has a shell-like or cap-like shape and can have a cylindrical outer contour. It serves for supporting the liner 10 and the stump and/or for stabilizing the distal end of the prosthesis socket 20. In addition, a device 25 for connecting a prosthetic component is arranged at the distal end of the prosthesis socket 20. The device 25 can be in the form, for example, of a receptacle of a pyramid adapter or in the form of a pyramid adapter itself.

A recess 27 is formed within the supporting device 26 in order to receive the closure system 40 in the mounted and fitted state such that no excessive pressure loadings occur at the end of the stump because of a central, punctiform introduction of force via the closure system 40. The closing cap 13 rests extensively on the beveled upper edge of the supporting device 26 and brings about a uniform distribution of pressure to the end of the stump.

A pass-through opening 24 for the tensioning means 30 is formed in the supporting device 26 and in the side wall 23 in the distal closing region 22 of the prosthesis socket. A seal 29 is arranged on the outer side of the pass-through opening 24 in order as far as possible to ensure fluid-tight sealing of the interior of the prosthesis socket 20. A blocking element 31 is arranged at the free end of the tensioning means 30 and is fastened releasably to the tensioning means 30 in order, after the tensioning means 30 is passed through the passage opening 24, to avoid said tensioning means being inadvertently pulled out during removing of the stump from the prosthesis socket 20.

A deflecting element 51 is arranged on the outer side of the side wall 23. The deflecting element 51 is in the form of an eyelet which is fastened to a mounting part which is arranged in a window 28 in the side wall 23 of the prosthesis socket. The free end of the tensioning means 30 is pulled through the deflecting element 51 when the fitted prosthetic liner 10 is coupled with the tensioning means 30 via the closure system 40. During entry into the prosthesis socket 20, the end of the tensioning means is at the same time pulled through the passage opening 24 such that the prosthetic liner 10 is pulled into the prosthesis socket 20. After the free end of the tensioning means 30 is passed through the deflecting element 51, the tensioning means 30 can be fixed on itself via fastening elements (not illustrated), for example hook and loop fasteners, loops or the like, and therefore, in the fully introduced state, the closure element 40 is subjected to a tensile stress which is applied by the tensioned tensioning means 30. Instead of a deflecting element, a second tensioning means or a fastening element can also be secured to the side wall and can be connected to the first tensioning means 30 in order to fix the first tensioning means 30 in the desired tensioned position.

In the mounted state, the closure system 40 is approximately level with the horizontally oriented passage opening 24, which is directed toward the lateral side, or is slightly above said passage opening, and therefore, when the tensioning means 30 is sufficiently tensioned, fixing of the prosthetic liner 10 within the socket 20 is ensured. Neither a lateral movement in the horizontal plane nor a pulling-out movement counter to the insertion direction of the prosthetic liner 10 is then possible. The closure system 40, which provides form-fitting locking by means of the two form-fitting elements 41, 42, blocks a lateral relative movement of the two form-fitting elements 41, 42 in the horizontal plane and also shifting in or counter to the insertion direction, and therefore only a rotational degree of freedom is available because of the spherical-head-shaped configuration of the second form-fitting element 42. All other degrees of freedom are blocked.

Alternatively to securing the tensioning means 30 on itself and deflecting same via a deflecting element 51, the tensioning means 30 can also be secured directly to the outer side of the side wall 23.

For removing the stump from the prosthesis socket 20, the fixing of the tensioning means 30 is released and the prosthetic liner 10 together with the stump is guided out of the prosthesis socket 20. The tensioning means 30 is also pulled out at the same time from the interior of the prosthesis socket 20. As soon as the prosthetic liner 10 is located outside the prosthesis socket 20, the tensioning means 30 can be released from the prosthetic liner 10, by release of the form-fitting elements 41, 42 from each other, for example by pressing a spring lock, and therefore the prosthesis socket 20 together with the remaining prosthesis components, which are fastened thereto, and the tensioning means 30 is separated from the prosthetic liner 10.

FIG. 2 shows a detailed illustration of the two form-fitting elements 41, 42 according to FIG. 1 in the locked state. The first form-fitting element 41 is fixed to the strap-like tensioning means 30. A spherical head is formed on the second form-fitting element 42, at the opposite end of which spherical head a screw thread is arranged, and therefore the second form-fitting element 42 is fastenable to the prosthetic liner 10 with a tool. The first form-fitting element 41 has a recess corresponding to the second form-fitting element 42, in which the spherical head can be snapped in order to lock the two form-fitting elements 41, 42 in a form-fitting manner to each other. For the mounting, the first form-fitting element 41 is pressed perpendicularly onto the spherical head, the material of the first form-fitting element 41 expands, or resilient components or slots are arranged therein and are shifted, and therefore, after the mounting, the spherical head is accommodated in the first form-fitting element 41. In the mounted state, only rotation of the first form-fitting element 41 about the longitudinal extent of the second form-fitting element 42 is possible; all other rotational or translational shifting is blocked.

In order to open the closure system and therefore in order to release the tensioning means 30 from the liner 10 (not illustrated), the first form-fitting element 40 is pulled off from the second form-fitting element 42 or separated by bending. Tools are not necessary for this.

A variant of the first form-fitting element 41 is shown in a side view and partial sectional illustration in FIG. 2a. The form-fitting element 41 has a disengaging mechanism 45 with which it is possible to disengage locking elements within the form-fitting element 41 by means of a pull on the outer collar, and therefore the second form-fitting element, which corresponds to a recess and is not illustrated, can be inserted without pressure or only under slight pressure into the recess of the first form-fitting element 41 and locked there. For the release, corresponding unlocking is undertaken by pulling on the collar 45.

FIG. 3 shows a variant of the closure system with the two form-fitting elements 41, 42 in the locked state. Instead of a spherical-head-shaped form-fitting element, locking is realized via a in the truncated-cone-like projection with an undercut and a corresponding recess in the first form-fitting element 41. In addition to the pure form-fitting lockings, magnetic securing 43 is arranged in the first form-fitting element 41 and pulls the second form-fitting element 42 into the end position and holds same therein or at least applies an additional holding force. In the locked state, all of the translational degrees of freedom are also blocked here. In the case of a truncated-cone-like configuration, a rotational degree of freedom is free, and the latter can also be blocked in the case of a configuration which is not rotationally symmetrical.

A further variant of the invention is illustrated schematically in FIG. 4. Instead of locking by placing on and pressing in the insertion direction of the prosthetic liner in the prosthesis socket, i.e. in the longitudinal extent of the prosthetic liner, in the variant according to FIG. 4 the locking takes place by pushing the first form-fitting element 41 into the second form-fitting element 42 in a horizontal plane, i.e. perpendicularly to the insertion direction of the prosthetic liner 10 into the prosthesis socket 20. After the insertion and displacing as far as an end stop, locking against an unintentional moving out can take place as a result of a resilient snap-action connection, a rotational movement or an additional form-fitting element or turning a lock.

After the first form-fitting element 41 is inserted by being pushed in the arrow direction into the second form-fitting element 42, form-fitting locking takes place, and therefore the tensioning means 30 is fixed to the prosthetic liner 10 and separating perpendicularly to the insertion direction of the prosthetic liner into the prosthesis socket is not possible. By means of the configuration of the second form-fitting element 42 with a solid side wall as end stop, further shifting after the end stop is reached is prevented.

FIG. 5 shows an enlarged detailed illustration of the closure system 40 with the two form-fitting elements 41, 42 and the locking direction. For unlocking purposes, pressure can be exerted on the first form-fitting element 41, as a result of which the corresponding form-fitting element is expanded and release counter to the insertion direction is made possible.

FIG. 6 shows a top view of the form-fitting elements 41, 42 according to FIG. 5. An insertion channel with an undercut is formed in the second form-fitting element 42. When the first form-fitting element 41 is inserted into the insertion channel, spring tongues are expanded and, after reaching the end position, surround a pin-shaped projection again and prevent unintentional unlocking, but permit pushing out by application of an oppositely directed force, optionally after actuation of an unblocking device.

Alternatively to the form-fitting elements shown, the latter can also be in the form of a hook and loop fastener. There are highly effective variants of hook and loop fasteners, with which it is possible to achieve a strong connection. Such high-strength hook and loop fasteners can also be used on account of the fact that a peeling-off movement is possible after at least partially removing the stump from the prosthesis socket 20. Pressure closures, pure sliding closures and/or quarter-turn fasteners, i.e. a sliding and rotating closure device can likewise be provided in order to connect the prosthetic liner 10 to the tensioning means 30 without a tool.

FIG. 7 shows a variant of the prosthesis socket system in the mounted state in a sectional illustration. The construction substantially corresponds to that of FIG. 1. It can be seen that the prosthetic liner 10 lies with its side wall 14 over substantially its entire longitudinal extent against the inner side of the side wall 23 of the prosthesis socket 20. A second tensioning means 35 is arranged instead of a deflecting element 51 within the window 28 in the side wall 23, said second tensioning means being able to be in the form of a strap, a lug or the like. For example, hook regions of a hook and loop fastener or components of identical effect are arranged on that side of the second tensioning means 35 which faces the socket 20 while the loop components or correspondingly acting fastening devices are arranged on the first tensioning means 30 on the outer side, i.e. the side facing away from the prosthesis socket 20. As a result, it is possible to connect the two tensioning means 30, 35 to each other in their end regions such that they can be secured to each other. Instead of a hook and loop fastener, other fastening devices 36 can also be arranged on the tensioning means 30, 35, for example a buckle, holes for hooks, bolts or other projections, press studs or clamping devices, with which the first tensioning means 30 can be secured to the second tensioning means 35.

FIG. 8 shows the variant according to FIG. 1 in the mounted state. A deflecting element 51 is fastened to the side wall 23 of the prosthesis socket 20. That end of the first tensioning means 30 which is led out of the prosthesis socket 20 is passed through the deflecting element 51. A component of a hook and loop fastener is arranged on the outer side of that portion of the tensioning means 30 which has not yet been passed through. The corresponding component of the hook and loop fastener is arranged on the turned-over side, which then points inward in the direction of the prosthesis socket, of the turned-over end of the first strap, and therefore, after the tensioning means 30 is turned over and deflected on itself, the tensioning means 30 in the form of a strap can be secured on itself via the devices 37 with the hook region and loop region.

A further variant of the invention is illustrated in FIG. 9, in which, in the exemplary embodiment illustrated, the tensioning means 30 is in turn fixed on itself, as shown in FIG. 8. In addition, a fixing device 50 in the form of a loop region or a hook region is arranged or formed on the outer side of the side wall 23 in order to be able to secure the tensioning means 30 on the outer wall either after deflection by the deflecting element 51, or else in order to be able to dispense with the deflecting element 51 and therefore also the window 28, and to fix the first tensioning means 30 directly to the side wall 23. Instead of fixing via a hook and loop fastener, said tensioning means can also be fixed via other form-fitting devices, for example by holes in the tensioning means 30 and one or more projections which latch into the holes, by buckles or by other form-fitting or force-fitting fixing devices 50.

FIG. 10a, in a detailed illustration, illustrates a tensioning means 30 which is secured in a form-fitting manner to the second form-fitting element 42 of a prosthetic liner (not illustrated specifically). In the exemplary embodiment illustrated, the tensioning means 30 is in the form of a strap which is turned back on itself and is in the form of two layers. An upper layer 33 and a lower layer 32 are connected at their free ends to a seam 392 via an end region. Alternatively or additionally, the two layers 32, 33 can be welded, adhesively bonded or connected to each other in some other way. In the front, turned-over region of the tensioning means 30, the two layers 32, 33 are likewise permanently connected to each other, for example via a seam 391, which can be in the form of an adhesive seam, welding seam or stitched seam. The connection of the two layers 32, 33 can be formed over the full surface area in the region of the free ends, and can be located over most of the full surface area or else basically also only at the circumference in the region of the turning-over point. A free region 38 in which the two layers 32, 33 are not connected to each other is formed between the two seam regions 391, 392, and therefore the two layers 32, 33 can be shifted away from each other in said central region.

A band 34 which can be in the form of a string, cable, cord, wire, strand or strap is fastened to the tensioning means 30. The band 34 projects beyond the turned-over end of the tensioning means 30 and, in the mounted state, surrounds the second form-fitting element 42 in such a manner that the tensioning means 30 cannot be released from the second form-fitting element either in the longitudinal extent of the undercut pin or transversely with respect thereto. The band 34 is fixed to the tensioning means 30 at two fastening points 341, 342, the first fastening point 341 is located in that region of the tensioning means 30 which lies section located on that side of the free region 38 which faces away from the second form-fitting element 42, while the second fastening point 342 is located in the region of the turning-over point of the tensioning means 30. It is also possible in principle for the second fastening point 342 to be located shifted further in the direction of the free region 38.

In the position according to FIG. 10a, the band portion which protrudes beyond the turned-over end of the tensioning means 30 is dimensioned in such a manner that the tensioning means 30 is pulled straight. The two layers 32, 33 also rest on each other in the free region 38, in which the two layers 32, 33 are not connected to each other via a seam, and the loop formed by the protruding band 34 substantially corresponds in respect of its circumference to the looped-around second form-fitting element 42 in order thus to fix the tensioning means 30 to the second form-fitting element 42.

The second form-fitting element 42 is in the form of a mushroom-shaped pin with a round cross section, and therefore the tensioning element 30 is movable about the longitudinal axis of the pin. In an alternative embodiment, it is possible to configure the pin to be non-circular or to provide same with a flattened portion in order not to permit any rotation in a plane perpendicular to the longitudinal axis of extent of the pin when the turned-over end of the tensioning means 30 lies against the flattened portion.

FIG. 10b illustrates the tensioning element 30 in a state in which the latter can be removed from the second form-fitting element 42. For this purpose, the front portion of the tensioning means 30, which portion is placed in the region of the turned-over point, moves in the direction of the rear portion with the free ends such that the tensioning means 30 is compressed. This results in compression in the free region 38, leading to two arches 382, 383 since the distance between the turned-over point of the tensioning means 30 and the free end is reduced. The band 34 is fixed at its rear end to the rear portion via the first fastening point 341 and in the front portion is guided in a longitudinally movable manner within the tensioning means 30 such that, during shifting of the turned-over point, the band 34 can slide out of the tensioning means 30, in particular when it is still fastened to the second form-fitting element 42. Only the front, turned-over end of the tensioning means 30 then has to be shifted in the arrow direction, as a result of which the loop which is formed by the band 34 outside the tensioning means 30 is enlarged. In the state according to FIG. 10b, it is possible to remove the loop formed by the band 34 in the region protruding beyond the turned-over point from the second form-fitting element 42. Alternatively to the embodiment illustrated, in which the band 34 is guided only along a channel or only with a portion in the front region of the double-layered tensioning means 30, it is also possible to design the band 34 as a tightening loop, in which the looping-around point of the tightening loop lies in the strap or tensioning means 30, namely on that side of the turned-over point between the upper layer 33 and the lower layer 32 which faces away from the second form-fitting element 42. The band 34 or the cable is led here out of the tensioning means 30 at the front end thereof and forms the loop which is placed around the second form-fitting element 42. Behind the front end of the tensioning means 30, the band 34 or the cable is placed in the form of a loop around that part of the band 34 or of the cable which is mounted movably in the front portion of the tensioning means 30, thus forming a tightening loop, in which, by pulling on the rear end of the tensioning means 30, the loop emerging from the tensioning means 30 is tightened. If, at the front end of the tensioning means 30, i.e. at the tensioning means portion in front of the free region 38, pulling is undertaken away from the second form-fitting element 42, the loop is enlarged and can be removed from the form-fitting element 42. Such a variant of the invention is shown in FIG. 10e.

The shortened, pushed-together state, as is reached after pulling back the front, turned-over end of the tensioning means 30, is shown in FIG. 10c. The arches 382, 383 are directed outward as free portions, and the band 34 forms a comparatively large loop which protrudes beyond the turned-over point. In order to reduce the loop diameter and therefore to secure the tensioning means 30 on the second form-fitting element 42 by the band 34 via the loop, the two ends of the tensioning means 30 are pulled apart again, which is shown in FIG. 10d and is indicated by the two arrows. By pulling apart the two ends of the tensioning means, the band 34 is also pulled through the front portion of the tensioning means and the free region 38, and therefore the loop diameter of the band 34 is reduced.

In an advantageous refinement, the tensioning means 30 is provided with resetting properties, for example by selection of material or by integrating resetting elements, such as plastics springs, struts, stiffening means or the like, such that automatic resetting into the state according to FIG. 10d takes place. As a result, a permanent prestressing force is applied to the band 34, and therefore the loop and the tensioning means 30 remain securely held on the second form-fitting element 42. Said prestressing force can be achieved by increased buckling stability of the material of the tensioning means.

1. A flexible prosthetic liner comprising:
a proximal access opening for a stump of a limb;
a distal end region;
a flexible tensioning member arranged on the prosthetic liner in the distal end region, wherein the tensioning member is releasably fastened to the prosthetic liner via a closure system, the closure system being actuable without a tool.

2. The prosthetic liner as claimed in claim 1, wherein the tensioning member is fastened to the prosthetic liner in at least one of a form-fitting and a force-fitting manner.

3. The prosthetic liner as claimed in claim 1, wherein a first form-fitting element is arranged on the tensioning member and enters into engagement with a second form-fitting element arranged on the prosthetic liner.

4. The prosthetic liner as claimed in claim 3, wherein the first and second form-fitting elements are in the form of at least one of a snap-action connection, a hook and loop fastener, a press stud, a sliding closure and a quarter-turn fastener.

5. The prosthetic liner as claimed in claim 3, wherein the closure system blocks the tensioning member counter to an insertion direction of the prosthetic liner from a prosthesis socket.

6. The prosthetic liner as claimed in claim 3, wherein the closure system blocks the tensioning member in at least one direction perpendicular to a pulling-out direction of the prosthetic liner from the prosthesis socket.

7. The prosthetic liner as claimed in claim 5, wherein an additional securing element for securing the prosthetic liner on the prosthesis socket is arranged at the distal end region of the prosthetic liner.

8. The prosthetic liner as claimed in claim 1, wherein the closure system has a magnetic securing feature.

9. A prosthesis socket system with a prosthetic liner as claimed in claim 1 and a prosthesis socket, the prosthesis socket having a proximal access opening, a side wall at least partially circumferentially surrounding the prosthetic liner, a distal closing region, devices for connecting a prosthetic component, and a pass-through opening for the tensioning member in the distal closing region.

10. The prosthesis socket system as claimed in claim 9, wherein a dimensionally stable supporting device with a recess for the closure system is arranged at the distal closing region.

11. The prosthesis socket system as claimed in claim 9, wherein a fixing device for the tensioning member or at least one deflecting element for the tensioning member, on which devices for securing same on itself are arranged, is arranged on an outer side of the side wall of the prosthesis socket.

12. The prosthesis socket system as claimed in claim 9, wherein a window is formed in a proximal region of the side wall for a second tensioning member or for securing a deflecting element.

13. The prosthesis socket system as claimed in claim 12, wherein the tensioning member and the second tensioning member have fastening devices for securing same to each other.

14. The prosthesis socket system as claimed in claim 9, wherein the pass-through opening is oriented perpendicular to the insertion direction of the prosthetic liner such that the first tensioning member can be brought laterally out of the prosthesis socket.

15. A flexible prosthetic liner comprising:
a flexible base body comprising:
an internal cavity;
a proximal access opening sized to receive a stump of a limb into the internal cavity;
a closed distal end region;
a flexible tensioning member arranged on the flexible base body in the distal end region;
a closure system releasably connecting the tensioning member to the flexible base body, the closure system being actuable without a tool.

16. The prosthetic liner as claimed in claim 15, wherein the tensioning member is fastened to the prosthetic liner in at least one of a form-fitting manner and a force-fitting manner.

17. The prosthetic liner as claimed in claim 15, wherein a first form-fitting element is arranged on the tensioning member and enters into engagement with a second form-fitting element arranged on the flexible base body.

18. The prosthetic liner as claimed in claim 17, wherein the first and second form-fitting elements are in the form of at least one of a snap-action connection, a hook and loop fastener, a press stud, a sliding closure and a quarter-turn fastener.

19. The prosthetic liner as claimed in claim 17, wherein the closure system blocks the tensioning member counter to an insertion direction of the prosthetic liner into a prosthesis socket.

20. The prosthetic liner as claimed in claim 17, wherein the closure system blocks the tensioning member in at least one direction perpendicular to a pulling-out direction of the prosthetic liner from a prosthesis socket.
